(19) United States
(12) Patent Application Publication
(10) Pub. No.: US 2015/0359724 A1
(43) Pub. Date: Dec. 17, 2015

(54) Acrylate Gel Nail Coating Compositions

(71) Applicant: Elementis Specialties, Inc., East Windsor, NJ (US)

(72) Inventors: Wouter Ijdo, Yardley, PA (US); Yanhui Chen, Plainsboro, NJ (US); Prashant Deshmukh, Plainsboro, NJ (US)

(21) Appl. No.: 14/479,932

(22) Filed: Sep. 8, 2014

Related U.S. Application Data
(60) Provisional application No. 62/035,038, filed on Aug. 8, 2014.
(60) Provisional application No. 62/012,733, filed on Jun. 16, 2014.
(60) Provisional application No. 62/012,732, filed on Jun. 16, 2014.

(51) Int. Cl. A61K 8/87; A61Q 3/02; A61K 8/73

(52) U.S. Cl. CPC A61K 8/87; A61K 8/731; A61Q 3/02

(57) ABSTRACT

A polymerizable nail coating composition includes a reactive (meth) acrylate monomer; a reactive (meth) acrylate terminated polymer selected from the group consisting of a reactive (meth) acrylate terminated polyol polymer, a reactive (meth) acrylate terminated polyester polymer, a reactive (meth) acrylate terminated polyol urethane polymer, a reactive (meth) acrylate terminated polyester polymer, and combinations thereof wherein said polymer is derived at least in part from a polyether or a polyester having either a melting point between 20° C. and 60° C. or a glass transition temperature between 20° C. and 60° C.; and a polymerization photoinitiator, wherein upon exposure to radiant energy, said polymerizable nail coating composition cures to a photocrosslinked polymer.

### CROSS-REFERENCE TO RELATED APPLICATIONS

This application claims priority benefit from U.S. Provisional Patent Application 62/012,732 filed Jun. 16, 2014; U.S. Provisional Patent Application 62/012,733 filed Jun. 16, 2014; and U.S. Provisional Patent Application 62/035,038 filed Aug. 8, 2014, each of which is incorporated by reference herein in its entirety.

### FIELD OF THE INVENTION

The present invention relates to polymerizable nail coating compositions exhibiting resistance to chipping and enhanced removal capability.

### BACKGROUND OF THE INVENTION

The use of radiation-curable gels in formation of nail enhancements or artificial nails has been an important part of the cosmetic industry since it was first introduced. U.S. Pat. No. 4,682,612, describing the use of actinic radiation-curable compositions suitable for preparation of artificial nails, is representative of this technology.

Ultra-violet radiation (UV) is the most conventional form of radiation used to cure gels in this art, however, visible light curing systems are also known. Professional nail technicians most typically apply UV curable gels designed for sculpting nails. Such UV-curable gels are usually composed of acrylic or methacrylic monomers and oligomers in a gel-like state that requires curing under a UV lamp. Such nail finishes can be applied directly to natural fingernails or toenails, or alternatively can be applied to nail extensions bonded to fingernails. In many cases, the artificial nails are coated with conventional nail polish after they are cured.

In addition, a considerable advantage of the use of the UV nail gel for the customer and the person performing the application is the reduced time needed to harden. A customer can spend up to an hour waiting for the solvent in nail enamel to evaporate, while the gel is set in 3 minutes or less. Disadvantageously, due to the crosslinked polymer which is formed while curing these gels they are much more difficult to remove than normal nail polishes. Thus, there is a need for compositions that give improved soak off capabilities.

### SUMMARY OF THE INVENTION

According to some embodiments of the present invention, a polymerizable nail coating composition includes a reactive (meth) acrylate monomer; a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof, wherein said reactive (meth) acrylate terminated polymer has at least two reactive acrylate groups, wherein said polymer is derived at least in part from a polyether or a polyester having either a melting point between 20° C. and 60° C. or a glass transition temperature between 20° C. and 60° C.; and a polymerization photoinitiator, wherein upon exposure to radiant energy, said polymerizable nail coating composition cures to a photocrosslinked polymer.

In some embodiments, after the coating composition is cured to form the photocrosslinked polymer on a nail surface, the photocrosslinked polymer is responsive to an external thermal stimuli such that the cured photocrosslinked polymer is removable from the nail surface in less than 10 minutes with exposure to an organic solvent, wherein the external thermal stimuli and organic solvent exposure are successive.

In certain embodiments, when the coating composition is cured to form the photocrosslinked polymer on a nail surface, the photocrosslinked polymer is removable from the nail surface with exposure to an organic solvent in an amount of time that is less than a time to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer.

In some embodiments, the reactive (meth) acrylate terminated polyether urethane polymer and/or reactive (meth) acrylate terminated polyester urethane polymer comprises monomer units derived from a monoisocyanate (meth) acrylate monomer and a polyether and/or polyester. The monoisocyanate (meth) acrylate monomer may be independently selected from the group consisting of 2-isocyanatoethyl methacrylate, 2-isocyanatoethyl acrylate, 3-isocyanatopropyl methacrylate, 1-methyl-2-isocyanatoethyl methacrylate and 1,1-dimethyl-2-isocyanatoethyl acrylate. The polyester may be independently selected from the group consisting of polycaprolactone diol, polycaprolactone, poly(D,L-lactide), poly (D,L-lactide-co-glycolide), and combinations thereof

In some embodiments, the reactive (meth) acrylate terminated polyether urethane polymer and/or reactive (meth) acrylate terminated polyester urethane polymer comprises monomer units derived from a polyisocyanate monomer, a (meth) acrylate monomer and a polyether and/or polyester. The polyisocyanate monomer may be independently selected from the group consisting of hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof. The polyester may be independently selected from the group consisting of polycaprolactone diol, polycaprolactone, poly(D,L-lactide), poly (D,L-lactide-co-glycolide), and combinations thereof

In certain embodiments, the reactive (meth) acrylate monomer is independently selected from the group consisting of hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

In some embodiments, the reactive (meth) acrylate terminated polyether urethane polymer and/or reactive (meth) acrylate terminated polyester urethane polymer is substantially free of unreacted isocyanate groups.

In some embodiments, the polymerizable nail coating composition further comprises a non-reactive solvent selected from the group consisting of acetone, ethyl acetate, butyl acetate, isopropyl alcohol, ethanol, methyl ethyl ketone, toluene, hexane, and mixtures thereof.

In certain embodiments, the polymerization photoinitiator is selected from the group consisting of benzoylphenylphosphinates, cyclohexylphenyl ketones, benzyl ketals, and mixtures thereof. In some embodiments, the polymerization photoinitiator is selected from the group consisting of 2,4,6-trimethylbenzoyldiphenylphosphinate, hydroxycyclohexyl phenyl ketone, benzyl dimethyl ketal, and mixtures thereof.

In some embodiments, the polymerizable nail coating composition further includes a keratin adhesion promoter. The keratin adhesion promoter may be selected from the group consisting of: nitrocellulose, cellulose acetopropionate, cellulose acetobutyrate and mixtures thereof.

In some embodiments, the external thermal stimuli has a temperature range of 20° C. to 65° C. for a time ranging from 5 minutes to 15 minutes.

According to some embodiments of the present invention, a polymerizable nail coating composition includes a reactive (meth) acrylate monomer; a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof, wherein said reactive (meth) acrylate terminated polymer has at least two reactive acrylate groups,; and a polymerization photoinitiator, wherein upon exposure to radiant energy, said polymerizable nail coating composition cures to a photocrosslinked polymer.

In some embodiments, the coating composition is cured to form the photocrosslinked polymer on a nail surface, the photocrosslinked polymer is removable from the nail surface with exposure to an organic solvent in an amount of time that is less than a time to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer.

In certain embodiments, the reactive (meth) acrylate terminated polyether urethane polymer and/or reactive (meth) acrylate terminated polyester urethane polymer comprises monomer units derived from a monoisocyanate (meth) acrylate monomer and a polyether and/or polyester. The monoisocyanate (meth) acrylate monomer may be independently selected from the group consisting of 2-isocyanatoethyl methacrylate, 2-isocyanatoethyl acrylate, 3-isocyanatopropyl methacrylate, 1-methyl-2-isocyanatoethyl methacrylate and 1,1-dimethyl-2-isocyanatoethyl acrylate. The polyether may be independently selected from the group consisting of polyoxypropylene glycol, polyoxyethylene glycol, poly(ethylene glycol)-poly(propylene glycol) block copolymer, poly(ethylene glycol)-block-poly(propylene glycol)-block-poly(ethylene glycol), poly(propylene glycol)-block-poly(ethylene glycol)-block poly(propylene glycol) copolymer and combinations thereof.

In some embodiments, the reactive (meth) acrylate terminated polyether urethane polymer and/or reactive (meth) acrylate terminated polyester urethane polymer comprises monomer units derived from a polyisocyanate monomer, a (meth) acrylate monomer and a polyether and/or polyester. The polyisocyanate monomer may be independently selected from the group consisting of hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof. The polyether may be independently selected from the group consisting of polyoxypropylene glycol, polyoxyethylene glycol, poly(ethylene glycol)-poly(propylene glycol) block copolymer, poly(ethylene glycol)-block-poly(propylene glycol)-block-poly(ethylene glycol), poly(propylene glycol)-block-poly(ethylene glycol)-block poly(propylene glycol) copolymer and combinations thereof.

In some embodiments, the reactive (meth) acrylate monomer is independently selected from the group consisting of hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

In certain embodiments, the reactive (meth) acrylate terminated polyether urethane polymer and/or reactive (meth) acrylate terminated polyester urethane polymer is substantially free of unreacted isocyanate groups.

In some embodiments, the polymerizable nail coating composition further comprises a non-reactive solvent selected from the group consisting of acetone, ethyl acetate, butyl acetate, isopropyl alcohol, ethanol, methyl ethyl ketone, toluene, hexane, and mixtures thereof.

In some embodiments, the polymerization photoinitiator is selected from the group consisting of benzoylphenylphosphinates, cyclohexylphenyl ketones, benzyl ketals, and mixtures thereof. The polymerization photoinitiator may be selected from the group consisting of 2,4,6-trimethylbenzoyldiphenylphosphinate, hydroxycyclohexyl phenyl ketone, benzyl dimethyl ketal, and mixtures thereof.

In certain embodiments, the polymerizable nail coating composition further comprises a keratin adhesion promoter. The keratin adhesion promoter may be selected from the group consisting of: nitrocellulose, cellulose acetopropionate, cellulose acetobutyrate and mixtures thereof.

### DETAILED DESCRIPTION OF THE EMBODIMENTS

### I. Polymerizable Nail Coating to Form a Photocrosslinked Polymer Which is Removed From a Nail With an External Thermal Stimuli and Solvent

In one embodiment, the present disclosure provides for a composition that adheres to the natural nail for periods in excess of two weeks and responds to an external thermal stimuli and organic solvent to ease removal, such that when the coating composition is cured to form a photocrosslinked polymer on a nail surface, the photocrosslinked polymer is removed from the nail surface with exposure to an organic solvent in an amount of time that is less than a time required to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer.

In one embodiment, the present invention provides for a polymerizable nail coating composition comprising: a reactive (meth) acrylate monomer; a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof, wherein said polymer is derived at least in part from a polyether and/or polyester having either a melting point between 20° C. and 60° C. or a glass transition temperature between 20° C. and 60° C.; and a polymerization photoinitiator, wherein upon exposure to radiant energy, said polymerizable nail coating composition cures to a photocrosslinked polymer. After the coating composition is cured to form the photocrosslinked polymer on a nail surface, the photocrosslinked polymer is responsive to an external thermal stimuli such that the cured photocrosslinked polymer is removed from the nail surface in less than 10 minutes with exposure to an organic solvent.

In some embodiments, the external thermal stimuli has a temperature range of: 20° C. to 65° C.; 30° C. to 55° C.; or 30° C. to 40° C., for a time ranging from 5 minutes to 15 minutes. In such embodiments, the cured photocrosslinked polymer may be removed from the nail surface in less than 10 minutes after successive exposure to the external thermal stimuli and an organic solvent. In some embodiments, less than 50% of the nail surface is covered with the cured photocrosslinked polymer following successive exposure to the external thermal stimuli and organic solvent. In some other embodiments, less than 25% of the nail surface is covered with the cured photocrosslinked polymer following successive exposure to the external thermal stimuli and organic solvent. In some other embodiments, less than 5% of the nail surface is covered with the cured photocrosslinked polymer following exposure to the external thermal stimuli and organic solvent.

In one embodiment, when the coating composition is cured to form the photocrosslinked polymer on a nail surface, the photocrosslinked polymer is removed from the nail surface with successive exposure to an external thermal stimuli and an organic solvent in an amount of time that is less than a time required to remove a comparative photocrosslinked polymer from a nail surface where the comparative photocrosslinked polymer was formed from a comparative polymerizable nail coating composition which does not contain a reactive acrylate terminated polyether urethane polymer and/or a reactive (meth) acrylate terminated polyester urethane polymer. In one such embodiment, the cured photocrosslinked polymer is removed in at least 50% of the time required to remove a comparative photocrosslinked polymer from a nail surface where the comparative photocrosslinked polymer was formed from a comparative polymerizable nail coating composition which does not contain a reactive acrylate terminated polyether urethane polymer and/or a reactive (meth) acrylate terminated polyester urethane polymer. In another such embodiment, the cured photocrosslinked polymer is removed in at least 25% of the time required to remove a comparative photocrosslinked polymer from a nail surface where the comparative photocrosslinked polymer was formed from a comparative polymerizable nail coating composition which does not contain a reactive acrylate terminated polyether urethane polymer and/or a reactive (meth) acrylate terminated polyester urethane polymer.

In one embodiment, when the coating composition is cured to form the photocrosslinked polymer on a nail surface, the amount of the photocrosslinked polymer which is removed from the nail surface with successive exposure to an external thermal stimuli and an organic solvent is greater in comparison to the amount removed of a comparative photocrosslinked polymer from a nail surface where the comparative photocrosslinked polymer was formed from a comparative polymerizable nail coating composition which does not contain a reactive acrylate terminated polyether urethane polymer and/or a reactive (meth) acrylate terminated polyester urethane polymer. In one such embodiment, the amount of photocrosslinked polymer removed by successive exposure to an external thermal stimuli and organic solvent is at least 50% greater than the amount of comparative photocrosslinked polymer. In another such embodiment, the amount of photocrosslinked polymer removed by successive exposure to an external thermal stimuli and organic solvent is at least 75% greater than the amount of comparative photocrosslinked polymer.

In some embodiments of the polymerizable nail coating composition, the reactive (meth) acrylate terminated urethane polymer and/or reactive (meth) acrylate terminated polyester urethane polymer includes monomer units derived from a monoisocyanate (meth) acrylate monomer and a polyether and/or polyester. In certain embodiments of the reactive (meth) acrylate terminated polyether urethane polymer and/or reactive (meth) acrylate terminated polyester urethane polymer, the monoisocyanate (meth) acrylate monomer is independently selected from 2-isocyanatoethyl methacrylate, 2-isocyanatoethyl acrylate, 3-isocyanatopropyl methacrylate, 1-methyl-2-isocyanatoethyl methacrylate, 1,1-dimethyl-2-isocyanatoethyl acrylate and combinations thereof. In certain embodiments of the reactive (meth) acrylate terminated polyesterurethane polymer, the polyester is independently selected from polycaprolactone diol, polycaprolactone, poly(D,L-lactide), poly (D,L-lactide-co-glycolide), and combinations thereof.

In some embodiments of the reactive (meth) acrylate terminated polyether urethane polymer and/or reactive (meth) acrylate terminated polyester urethane polymer, the reactive (meth) acrylate terminated polyether urethane polymer and/or reactive (meth) acrylate terminated polyester urethane polymer is substantially free of unreacted isocyanate groups.

In some other embodiments of the polymerizable nail coating composition, the reactive (meth) acrylate terminated polyether urethane polymer and/or reactive (meth) acrylate terminated polyester urethane polymer comprises monomer units derived from a polyisocyanate monomer, a (meth) acrylate monomer and a polyether and/or polyester. In certain embodiments of the reactive (meth) acrylate terminated polyether urethane polymer and/or reactive (meth) acrylate terminated polyester urethane polymer, the polyisocyanate monomer is independently selected from hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof. In certain embodiments of the reactive (meth) acrylate terminated polyester urethane polymer, the polyester is independently selected from polycaprolactone diol, polycaprolactone, poly(D,L-lactide), poly (D,L-lactide-co-glycolide), and combinations thereof. In some embodiments, a polyol may be used independently selected from the group consisting of trimethylolethane, trimethylolpropane, trimethylolbutane, neopentyl glycol and pentaerythritol.

In some embodiments of the reactive (meth) acrylate terminated polyether urethane polymer and/or reactive (meth) acrylate terminated polyester urethane polymer, the reactive (meth) acrylate terminated polyether urethane polymer and/or reactive (meth) acrylate terminated polyester urethane polymer is substantially free of unreacted isocyanate groups.

In some embodiments of the polymerizable nail coating composition, the reactive (meth) acrylate monomer is independently selected from hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

In certain embodiments, the polymerizable nail coating composition may further include a crosslinking compound. Such crosslinking compound may be independently selected from di-HEMA trimethyl hexyl dicarbamate, 1,4-butane diol di(meth)acrylate, ethylene glycol di(meth)acrylate, triethylene glycol di(meth)acrylate, polyethylene glycol di(meth)acrylate, polypropylene glycol di(meth)acrylate and combinations thereof.

In certain embodiments, the polymerizable nail coating composition may further include a non-reactive solvent such as acetone, ethyl acetate, butyl acetate, isopropyl alcohol, ethanol, methyl ethyl ketone, toluene, hexane, and mixtures thereof.

In certain embodiments, the polymerizable nail coating composition may include a polymerization photoinitiator is selected from benzoylphenylphosphinates, cyclohexylphenyl ketones, benzyl ketals, and mixtures thereof. In some such embodiments, the polymerization photoinitiator is selected from 2,4,6-trimethylbenzoyldiphenylphosphinate, hydroxycyclohexyl phenyl ketone, benzyl dimethyl ketal, and mixtures thereof.

In certain embodiments, the polymerizable nail coating composition may include a keratin adhesion promoter. In some such embodiments, the keratin adhesion promoter is selected from nitrocellulose, cellulose acetopropionate, cellulose acetobutyrate and mixtures thereof. In some such embodiments, the keratin adhesion promoter may include polyvinylbutyral and/or tosylamide formaldehyde resins.

### II. Polymerizable Nail Coating to Form a Photocrosslinked Polymer Which is Removed From a Nail With Solvent

In one embodiment, the present invention provides for a polymerizable nail coating composition comprising: a reactive (meth) acrylate monomer; a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof; and a polymerization photoinitiator, wherein upon exposure to radiant energy, said polymerizable nail coating composition cures to a photocrosslinked polymer. After the coating composition is cured to form the photocrosslinked polymer on a nail surface, the photocrosslinked polymer may be removed from the nail surface in less than 10 minutes with exposure to an organic solvent.

In one embodiment, when the coating composition is cured to form the photocrosslinked polymer on a nail surface, the photocrosslinked polymer is removed from the nail surface with exposure to an organic solvent in an amount of time that is less than a time required to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer. In one such embodiment, the cured photocrosslinked polymer is removed in at least 50% of the time required to remove a comparative photocrosslinked polymer from a nail surface. In another such embodiment, the cured photocrosslinked polymer is removed in at least 25% of the time required to remove a comparative photocrosslinked polymer from a nail surface.

In one embodiment, when the coating composition is cured to form the photocrosslinked polymer on a nail surface, the amount of the photocrosslinked polymer which is removed from the nail surface with exposure to an organic solvent is greater in comparison to the amount removed of a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer. In one such embodiment, the amount of photocrosslinked polymer removed by exposure to an organic solvent is at least 50% greater than the amount of comparative photocrosslinked polymer. In another such embodiment, the amount of photocrosslinked polymer removed by exposure to an organic solvent is at least 75% greater than the amount of comparative photocrosslinked polymer.

In some embodiments of the polymerizable nail coating composition, the reactive (meth) acrylate terminated polyether urethane polymer and/or reactive (meth) acrylate terminated polyester urethane polymer includes monomer units derived from a monoisocyanate (meth) acrylate monomer and a polyether and/or polyester. In certain embodiments of the reactive (meth) acrylate terminated polyether urethane polymer and/or reactive (meth) acrylate terminated polyester urethane polymer, the monoisocyanate (meth) acrylate monomer is independently selected from 2-isocyanatoethyl methacrylate, 2-isocyanatoethyl acrylate, 3-isocyanatopropyl methacrylate, 1-methyl-2-isocyanatoethyl methacrylate, 1,1-dimethyl-2-isocyanatoethyl acrylate and combinations thereof. In certain embodiments of the reactive (meth) acrylate terminated polyether urethane polymer, the polyether is independently selected from polyoxypropylene glycol, polyoxyethylene glycol, poly(ethylene glycol)-polypropylene glycol) block copolymer, poly(ethylene glycol)-block-polypropylene glycol)-block-poly(ethylene glycol), poly(propylene glycol)-block-poly(ethylene glycol)-block poly(propylene glycol) copolymer, polyester polyol, polyether polyol and combinations thereof.

In some embodiments of the reactive (meth) acrylate terminated polyether urethane polymer and/or reactive (meth) acrylate terminated polyester urethane polymer, the reactive (meth) acrylate terminated polyether urethane polymer and/or reactive (meth) acrylate terminated polyester urethane polymer is substantially free of unreacted isocyanate groups.

In some other embodiments of the polymerizable nail coating composition, the reactive (meth) acrylate terminated polyether urethane polymer and/or reactive (meth) acrylate terminated polyester urethane polymer comprises monomer units derived from a polyisocyanate monomer, a (meth) acrylate monomer and a polyether and/or polyester. In certain embodiments of the reactive (meth) acrylate terminated polyether urethane polymer and/or reactive (meth) acrylate terminated polyester urethane polymer, the polyisocyanate monomer is independently selected from hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof. In certain embodiments of the reactive (meth) acrylate terminated polyether urethane polymer, the polyether is independently selected from polyoxypropylene glycol, polyoxyethylene glycol, poly(ethylene glycol)-poly(propylene glycol) block copolymer, poly(ethylene glycol)-block-poly(propylene glycol)-block-poly(ethylene glycol), poly(propylene glycol)-block-poly(ethylene glycol)-block poly(propylene glycol) copolymer and combinations thereof. In some embodiments, a polyol may be used independently selected from the group consisting of trimethylolethane, trimethylolpropane, trimethylolbutane, neopentyl glycol and pentaerythritol.

In some embodiments of the reactive (meth) acrylate terminated polyether urethane polymer and/or reactive (meth) acrylate terminated polyester urethane polymer, the reactive (meth) acrylate terminated polyether urethane polymer and/or reactive (meth) acrylate terminated polyester urethane polymer is substantially free of unreacted isocyanate groups.

In some embodiments of the polymerizable nail coating composition, the reactive (meth) acrylate monomer is independently selected from hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

In certain embodiments, the polymerizable nail coating composition may further include a crosslinking compound. Such crosslinking compound may be independently selected from di-HEMA trimethyl hexyl dicarbamate, 1,4-butane diol di(meth)acrylate, ethylene glycol di(meth)acrylate, triethylene glycol di(meth)acrylate, polyethylene glycol di(meth)acrylate, polypropylene glycol di(meth)acrylate and combinations thereof.

In certain embodiments, the polymerizable nail coating composition may further include a non-reactive solvent such as acetone, ethyl acetate, butyl acetate, isopropyl alcohol, ethanol, methyl ethyl ketone, toluene, hexane, and mixtures thereof.

In certain embodiments, the polymerizable nail coating composition may include a polymerization photoinitiator is selected from benzoylphenylphosphinates, cyclohexylphenyl ketones, benzyl ketals, and mixtures thereof. In some such embodiments, the polymerization photoinitiator is selected from 2,4,6-trimethylbenzoyldiphenylphosphinate, hydroxycyclohexyl phenyl ketone, benzyl dimethyl ketal, and mixtures thereof.

In certain embodiments, the polymerizable nail coating composition may include a keratin adhesion promoter. In some such embodiments, the keratin adhesion promoter is selected from nitrocellulose, cellulose acetopropionate, cellulose acetobutyrate and mixtures thereof. In some such embodiments, the keratin adhesion promoter may include polyvinylbutyral and/or tosylamide formaldehyde resins.

The inventive compositions, described herein, may be polymerizable with actinic radiation. The actinic radiation may be ultraviolet (UV) radiation. The UV radiation may be characterized by wavelengths of between 210 nanometers to 600 nanometers; 250 nanometers to 420 nanometers; and 350 to 410 nanometers. In some embodiments, the actinic radiation may be provided by a LED lamp or a UV lamp.

The compositions disclosed herein may be applied directly to the natural nail surface, with no pretreatment of the nail surface or application of a base or primer coat. However, it may be helpful to clean the nail surface of any residual oils before applying the curable composition. This may be accomplished, for example, by wiping the nail surface with a suitable solvent capable of dissolving the oils. Once a layer of the composition has been applied (typically, over the entire surface of an individual nail), the layer is exposed to energy (e.g., actinic radiation such as UV or visible light) for a time effective to cause polymerization (curing) of the energy-curable components of the composition. The compositions disclosed herein may be applied as part of a color coating composition.

### EXAMPLES

The following examples further describe and demonstrate illustrative embodiments within the scope of the present invention. The examples are given solely for illustration and are not to be construed as limitations of this invention as many variations are possible without departing from the spirit and scope thereof.

### Example 1

### Synthesis of polycaprolactone-urethane dimethacrylate

The reaction is carried out neat (no solvent). A polycaprolactone diol, having a Mₙ=2 kg/mol, was placed into reaction vial with mechanical stirrer. The reactor is attached with rubber septa and nitrogen is purged for 60 minutes while simultaneously the reactor was heated to 90° C. 2-isocyanatoethyl methacrylate was then added using syringe to the reaction mixture under constant stirring and catalyst amount of bismuth catalyst (K-KAT348) was added using syringe. The polycaprolactone diol and isocyanatoethyl methacrylate were used at a mole ratio of 1:1.98. The reaction was carried out at 90° C. for one hour. The reaction mixture was cooled down and precipitated in methanol and dried in vacuum for overnight.

### Example 2

### Synthesis of PEG-PPG-PEG-urethane dimethacrylate

A polyethyleneglycol-polypropylene glycol-polyethyelene glycol copolymer (“PEG-PPG-PEG”), having a Mₙ=2.8 kg/mol, was placed into reaction vial with mechanical stirrer. Pluronic® L-81 is a commercial source of the PEG-PPG-PEG copolymer. The reactor is attached with rubber septa and nitrogen is purged for 60 minutes while simultaneously the reactor was heated to 90° C. 2-isocyanatoethyl methacrylate was then added using syringe to the reaction mixture under constant stirring and catalyst amount of bismuth catalyst (K-KAT348) was added using syringe. The PEG-PPG-PEG copolymer and isocyanatoethyl methacrylate were used at a mole ratio of 1:1.98. The reaction was carried out at 90° C. for one hour in the absence of solvent. The reaction mixture was cooled to room temperature. The resulting PEG-PPG-PEG urethane dimethacrylate copolymer was a liquid at room temperature.

### Example 3

### Synthesis of PPG-PEG-PPG-urethane dimethacrylate

A polypropyleneglycol-polyethylene glycol-polypropylene glycol copolymer (“PPG-PEG-PPG”), having a Mₙ=2.7 kg/mol, was placed into reaction vial with mechanical stirrer. Pluronic® 17R4 is a commercial source of the PEG-PPG-PEG copolymer. The reactor is attached with rubber septa and nitrogen is purged for 60 minutes while simultaneously the reactor was heated to 90° C. 2-isocyanatoethyl methacrylate was then added using syringe to the reaction mixture under constant stirring and catalyst amount of bismuth catalyst (K-KAT348) was added using syringe. The PEG-PPG-PEG copolymer and isocyanatoethyl methacrylate were used at a mole ratio of 1:1.98. The reaction was carried out at 90° C. for one hour in the absence of solvent. The reaction mixture was cooled to room temperature. The resulting PEG-PPG-PEG urethane dimethacrylate copolymer was a liquid at room temperature.

### Example 4

### Synthesis of polycaprolactone-urethane dimethacrylate

A polycaprolactone diol, Mₙ=2 kg/mol, was placed into reaction vial with mechanical stirrer. The reactor was attached with rubber septa and nitrogen is purged for 60 minutes and simultaneously the reactor was heated to 75° C. Isophoron diisocyanate (“IPDI”) and 2-hydroxy ethyl methacrylate (“HEMA”) were added using syringe to the reaction mixture under constant stirring and catalyst amount of bismuth catalyst (K-KAT348) is added using syringe. The polycaprolactone diol, IPDI and HEMA were used in mole ratios of 1:2:2.1. The reaction was performed at 75° C. for 45 minutes. The reaction mixture was cooled to room temperature. The recovered reaction product contained 70 wt. % polycaprolactone urethane dimethacrylate, 10 wt. % HEMA and 20 wt. % Bis-HEMA IPDI urethane.

### Example 5

### Synthesis of PEG-PPG-PEG-urethane dimethacrylate

A PEG-PPG-PEG copolymer, Mₙ=2.8 kg/mol, was placed into reaction vial with mechanical stirrer. Pluronic® L-81 is a commercial source of the PEG-PPG-PEG copolymer. The reactor was attached with rubber septa and nitrogen is purged for 60 minutes and simultaneously the reactor was heated to 75° C. Isophoron diisocyanate (“IPDI”) and 2-hydroxy ethyl methacrylate (“HEMA”) were added using syringe to the reaction mixture under constant stirring and catalyst amount of bismuth catalyst (K-KAT348) is added using syringe. The PEG-PPG-PEG copolymer, IPDI and HEMA were used in mole ratios of 1:2:2.1. The reaction was performed at 75° C. for 45 minutes. The reaction mixture was cooled to room temperature. The recovered reaction product contained 70 wt. % PEG-PPG-PEG-urethane dimethacrylate, 10 wt. % HEMA and 20 wt. % Bis-HEMA IPDI urethane.

### Example 6

### Synthesis of PPG-PEG-PPG-urethane dimethacrylate

A PPG-PEG-PPG copolymer, Mₙ=2.7 kg/mol, was placed into reaction vial with mechanical stirrer. Pluronic® 17R4 is a commercial source of the PEG-PPG-PEG copolymer. The reactor was attached with rubber septa and nitrogen is purged for 60 minutes and simultaneously the reactor was heated to 75° C. Isophoron diisocyanate (“IPDI”) and 2-hydroxy ethyl methacrylate (“HEMA”) were added using syringe to the reaction mixture under constant stirring and catalyst amount of bismuth catalyst (K-KAT348) is added using syringe. The PEG-PPG-PEG copolymer, IPDI and HEMA were used in mole ratios of 1:2:2.1. The reaction was performed at 75° C. for 45 minutes. The reaction mixture was cooled to room temperature. The recovered reaction product contained 70 wt. % PEG-PPG-PEG-urethane dimethacrylate, 10 wt. % HEMA and 20 wt. % Bis-HEMA IPDI urethane.

### Example 7

The polycaprolactone-urethane dimethacrylate, of Example 1, was mixed with a commercial nail gel base formulation to form a base coating. The base coating was applied to glass slides, cured for 30 seconds using UV light. A color coat was then applied using a commercial nail coloring formulation and cured for 30 seconds using UV light. A top coat was then applied using a commercial top coat formulation and cured for 30 seconds using UV light and then aged for one week at room temperature. The coated glass slides were then heated at 60° C. for two minutes followed by acetone soak for 4 minutes.

### Comparative Example

A commercial nail gel base formulation was applied to glass slides, cured for 30 seconds using UV light. A color coat was then applied using a commercial nail coloring formulation and cured for 30 seconds using UV light. A top coat was then applied using a commercial top coat formulation and cured for 30 seconds using UV light and then aged for one week at room temperature. The coated glass slides were then heated at 60° C. for two minutes followed by acetone soak for 4 minutes. The results of Example 3 and Comparative Example are shown below.

TABLE 1

| | | % Coating Remaining on |
|---|---|---|
| Wt. % Polymer | Wt. & Base | Slide after heat and |
| and Example | Formulation | acetone treatment |
| 10 wt. % of Example 1 | 90 | <2% |
| 20 wt. % of Example 1 | 80 | <2% |
| 30 wt. % of Example 1 | 70 | <5% |
| 10 wt. % of Comparative | 90 | 100% |
| Example | | |

The present disclosure may be embodied in other specific forms without departing from the spirit or essential attributes of the invention. Accordingly, reference should be made to the appended claims, rather than the foregoing specification, as indicating the scope of the disclosure. Although the foregoing description is directed to the preferred embodiments of the disclosure, it is noted that other variations and modification will be apparent to those skilled in the art, and may be made without departing from the spirit or scope of the disclosure.

What is claimed:

1. A polymerizable nail coating composition comprising
a reactive (meth) acrylate monomer;
a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof, wherein said reactive (meth) acrylate terminated polymer has at least two reactive acrylate groups, wherein said polymer is derived at least in part from a polyether or a polyester having either a melting point between 20° C. and 60° C. or a glass transition temperature between 20° C. and 60° C.; and
a polymerization photoinitiator, wherein upon exposure to radiant energy, said polymerizable nail coating composition cures to a photocrosslinked polymer.

2. The polymerizable nail coating composition according to claim 1, wherein after the coating composition is cured to form the photocrosslinked polymer on a nail surface, the photocrosslinked polymer is responsive to an external thermal stimuli such that the cured photocrosslinked polymer is removable from the nail surface in less than 10 minutes with exposure to an organic solvent, wherein the external thermal stimuli and organic solvent exposure are successive.

3. The polymerizable nail coating composition according to claim 1, wherein when the coating composition is cured to form the photocrosslinked polymer on a nail surface, the photocrosslinked polymer is removable from the nail surface with exposure to an organic solvent in an amount of time that is less than a time to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer.

4. The polymerizable nail coating composition according to claim 1, wherein the reactive (meth) acrylate terminated polyester urethane polymer comprises monomer units derived from a monoisocyanate (meth) acrylate monomer and a polyester.

5. The polymerizable nail coating composition according to claim 4, wherein the monoisocyanate (meth) acrylate monomer is independently selected from the group consisting of 2-isocyanatoethyl methacrylate, 2-isocyanatoethyl acrylate, 3-isocyanatopropyl methacrylate, 1-methyl-2-isocyanatoethyl methacrylate and 1,1-dimethyl-2-isocyanatoethyl acrylate.

6. The polymerizable nail coating composition according to claim 5, wherein the polyester is independently selected from the group consisting of polycaprolactone diol, polycaprolactone, poly(D,L-lactide), poly (D,L-lactide-co-glycolide), and combinations thereof.

7. The polymerizable nail coating composition according to claim 1, wherein the reactive (meth) acrylate terminated polyester urethane polymer comprises monomer units derived from a polyisocyanate monomer, a (meth) acrylate monomer and a polyester.

8. The polymerizable nail coating composition according to claim 7, wherein the polyisocyanate monomer is independently selected from the group consisting of hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof.

9. The polymerizable nail coating composition according to claim 8, wherein the polyester is independently selected from the group consisting of polycaprolactone diol, polycaprolactone, poly(D,L-lactide), poly (D,L-lactide-co-glycolide), and combinations thereof.

10. The polymerizable nail coating composition according to claim 1, wherein the reactive (meth) acrylate monomer is independently selected from the group consisting of hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

11. The polymerizable nail coating composition according to claim 1, wherein the reactive (meth) acrylate terminated polyester urethane polymer is substantially free of unreacted isocyanate groups.

12. The polymerizable nail coating composition according to claim 1, further comprising a non-reactive solvent selected from the group consisting of acetone, ethyl acetate, butyl acetate, isopropyl alcohol, ethanol, methyl ethyl ketone, toluene, hexane, and mixtures thereof.

13. The polymerizable nail coating composition according to claim 1, wherein the polymerization photoinitiator is selected from the group consisting of benzoylphenylphosphinates, cyclohexylphenyl ketones, benzyl ketals, and mixtures thereof.

14. The polymerizable nail coating composition according to claim 13, wherein the polymerization photoinitiator is selected from the group consisting of 2,4,6-trimethylbenzoyldiphenylphosphinate, hydroxycyclohexyl phenyl ketone, benzyl dimethyl ketal, and mixtures thereof.

15. The polymerizable nail coating composition according to claim 1, further comprising a keratin adhesion promoter.

16. The polymerizable nail coating composition according to claim 15, where in the keratin adhesion promoter is selected from the group consisting of: nitrocellulose, cellulose acetopropionate, cellulose acetobutyrate and mixtures thereof.

17. The polymerizable nail coating composition according to claim 1, wherein the external thermal stimuli has a temperature range of 20° C. to 65° C. for a time ranging from 5 minutes to 15 minutes.

18. A polymerizable nail coating composition comprising
a reactive (meth) acrylate monomer;
a reactive (meth) acrylate terminated polymer selected from the group consisting of a reactive (meth) acrylate terminated polyol polymer, a reactive (meth) acrylate terminated polyester polymer, a reactive (meth) acrylate terminated polyether polymer, a reactive (meth) acrylate terminated polyol urethane polymer, a reactive (meth) acrylate terminated polyester urethane polymer, a reactive (meth) acrylate terminated polyether urethane polymer, a reactive (meth) acrylate terminated polyether/polyester urethane polymer and combinations thereof, wherein said reactive (meth) acrylate terminated polymer has at least two reactive acrylate groups,; and
a polymerization photoinitiator, wherein upon exposure to radiant energy, said polymerizable nail coating composition cures to a photocrosslinked polymer.

19. The polymerizable nail coating composition according to claim 18, wherein when the coating composition is cured to form the photocrosslinked polymer on a nail surface, the photocrosslinked polymer is removable from the nail surface with exposure to an organic solvent in an amount of time that is less than a time to remove a comparative photocrosslinked polymer from a nail surface where the comparative photocrosslinked polymer was formed from a comparative polymerizable nail coating composition which does not contain a reactive (meth) acrylate terminated polyether urethane polymer and/or a reactive (meth) acrylate terminated polyester urethane polymer.

20. The polymerizable nail coating composition according to claim 18, wherein the reactive (meth) acrylate terminated polyether urethane polymer comprises monomer units derived from a monoisocyanate (meth) acrylate monomer and a polyether.

21. The polymerizable nail coating composition according to claim 20, wherein the monoisocyanate (meth) acrylate monomer is independently selected from the group consisting of 2-isocyanatoethyl methacrylate, 2-isocyanatoethyl acrylate, 3-isocyanatopropyl methacrylate, 1-methyl-2-isocyanatoethyl methacrylate and 1,1-dimethyl-2-isocyanatoethyl acrylate.

22. The polymerizable nail coating composition according to claim 21, wherein the polyether is independently selected from the group consisting of polyoxypropylene glycol, polyoxyethylene glycol, poly(ethylene glycol)-poly(propylene glycol) block copolymer, poly(ethylene glycol)-block-poly(propylene glycol)-block-poly(ethylene glycol), poly(propylene glycol)-block-poly(ethylene glycol)-block poly(propylene glycol) copolymer and combinations thereof and combinations thereof.

23. The polymerizable nail coating composition according to claim 18, wherein the (meth) reactive acrylate terminated polyether urethane polymer comprises monomer units derived from a polyisocyanate monomer, a (meth) acrylate monomer and a polyether.

24. The polymerizable nail coating composition according to claim 23, wherein the polyisocyanate monomer is independently selected from the group consisting of hexamethylene diisocyanate, trimethyl hexamethylene diisocyanate, isophorone diisocyanate, tetramethyl xylylene diisocyanate, 4,4′-methylene bis(cyclohexylisocyanate), toluene diisocyanate, diphenylmethane diisocyanate and combinations thereof.

25. The polymerizable nail coating composition according to claim 24, wherein the polyether is independently selected from the group consisting of polyoxypropylene glycol, polyoxyethylene glycol, poly(ethylene glycol)-poly(propylene glycol) block copolymer, poly(ethylene glycol)-block-poly(propylene glycol)-block-poly(ethylene glycol), poly(propylene glycol)-block-poly(ethylene glycol)-block poly(propylene glycol) copolymer and combinations thereof.

26. The polymerizable nail coating composition according to claim 18, wherein the reactive (meth) acrylate monomer is independently selected from the group consisting of hydroxyethylmethacrylate (HEMA), hydroxypropylmethacrylate (HPMA), ethyl methacrylate (EMA), tetrahydrofurfuryl methacrylate (THFMA), pyromellitic dianhydride di(meth)acrylate, pyromellitic dianhydride glyceryl dimethacrylate, pyromellitic dimethacrylate, methacroyloxyethyl maleate, 2-hydroxyethyl methacrylate/succinate, 1,3-glycerol dimethacrylate/succinate adduct, phthalic acid monoethyl methacrylate, acetoacetoxy ethyl methacylate (AAEMA), and mixtures thereof.

27. The polymerizable nail coating composition according to claim 18, wherein the reactive (meth) acrylate terminated polyether urethane polymer is substantially free of unreacted isocyanate groups.

28. The polymerizable nail coating composition according to claim 18, further comprising a non-reactive solvent selected from the group consisting of acetone, ethyl acetate, butyl acetate, isopropyl alcohol, ethanol, methyl ethyl ketone, toluene, hexane, and mixtures thereof.

29. The polymerizable nail coating composition according to claim 18, wherein the polymerization photoinitiator is selected from the group consisting of benzoylphenylphosphinates, cyclohexylphenyl ketones, benzyl ketals, and mixtures thereof.

30. The polymerizable nail coating composition according to claim 29, wherein the polymerization photoinitiator is selected from the group consisting of 2,4,6-trimethylbenzoyldiphenylphosphinate, hydroxycyclohexyl phenyl ketone, benzyl dimethyl ketal, and mixtures thereof.

31. The polymerizable nail coating composition according to claim 18, further comprising a keratin adhesion promoter.

32. The polymerizable nail coating composition according to claim 31, wherein the keratin adhesion promoter is selected from the group consisting of: nitrocellulose, cellulose acetopropionate, cellulose acetobutyrate and mixtures thereof.
